(19) United States
(12) Patent Application Publication
(10) Pub. No.: US 2014/0171769 A1
(43) Pub. Date: Jun. 19, 2014

(54) SYSTEMS AND METHODS FOR DISTINGUISHING BETWEEN CENTRAL APNEA AND OBSTRUCTIVE APNEA

(71) Applicant: COVIDIEN LP, Mansfield, MA (US)

(72) Inventors: James Ochs, Seattle, WA (US); Scott Amundson, Oakland, CA (US)

(73) Assignee: Covidien LP, Mansfield, MA (US)

(21) Appl. No.: 13/719,175

(22) Filed: Dec. 18, 2012

(51) Int. Cl. A61B 5/00; A61B 5/0205; A61B 5/1455

(52) U.S. Cl. CPC A61B 5/4818; A61B 5/14551; A61B 5/7278; A61B 5/7282; A61B 5/7225; A61B 5/0205; A61B 5/746
USPC 600/324

(57) ABSTRACT

A patient monitoring system may acquire a time series of oxygen saturation data based on a physiological signal. A potential apneic event may be detected in the time series of oxygen saturation data in the form of a desaturation followed by a resaturation defined by a fall peak, nadir, and rise peak crossing respective thresholds. The potential apneic event may be qualified using a plurality of metrics derived from a portion of the time series of oxygen saturation data that corresponds to the potential apneic event. The qualified apneic event may be classified as being due to one of central apnea and obstructive apnea based on the output of a classification neural network the inputs to which comprise at least a second plurality of metrics.

The present disclosure relates to physiological signal processing, and more particularly relates to distinguishing between central apnea and obstructive apnea.

### SUMMARY

A method for distinguishing between central apnea and obstructive apnea comprises detecting a potential apneic event in a time series of oxygen saturation data, the potential apneic event being in the form of a desaturation followed by a resaturation defined by a fall peak, nadir, and rise peak crossing respective thresholds. The potential apneic event may be qualified as a qualified apneic event using a first plurality of metrics derived from a portion of the time series of oxygen saturation data that corresponds to the potential apneic event. The qualified apneic event may be classified as being due to one of central apnea and obstructive apnea based on the output of a classification neural network the inputs to which comprise at least a second plurality of metrics.

A non-transitory computer-readable storage medium for use in classifying an apneic event may have computer program instructions recorded thereon for detecting a potential apneic event in a time series of oxygen saturation data, the potential apneic event being in the form of a desaturation followed by a resaturation defined by a fall peak, nadir, and rise peak crossing respective thresholds. The computer-readable storage medium may have computer program instructions for qualifying the potential apneic event as a qualified apneic event using a first plurality of metrics derived from a portion of the time series of oxygen saturation data that corresponds to the potential apneic event. The computer-readable storage medium may have computer program instructions for classifying the qualified apneic event as being due to one of central apnea and obstructive apnea based on the output of a classification neural network the inputs to which comprise at least a second plurality of metrics.

A patient monitoring system comprises processing equipment configured to detect a potential apneic event in a time series of oxygen saturation data, the potential apneic event being in the form of a desaturation followed by a resaturation defined by a fall peak, nadir, and rise peak crossing respective thresholds. The processing equipment is configured to qualify the potential apneic event as a qualified apneic event using a first plurality of metrics derived from a portion of the time series of oxygen saturation data that corresponds to the potential apneic event. The processing equipment is configured to classify the qualified apneic event as being due to one of central apnea and obstructive apnea based on the output of a classification neural network the inputs to which comprise at least the second plurality of metrics.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 is a flow diagram showing illustrative steps for distinguishing between central apnea and obstructive apnea in accordance with some embodiments of the present disclosure;

FIG. 4 is a graph showing illustrative reciprocations in accordance with some embodiments of the present disclosure;

FIG. 5 is a series of graphs showing an illustrative time series of oxygen saturation data trend data, an upper oxygen saturation threshold, and a lower oxygen saturation threshold in accordance with some embodiments of the present disclosure;

FIG. 6 is an illustrative neural network for qualifying a reciprocation in accordance with some embodiments of the present disclosure;

FIG. 7 is a series of graphs showing illustrative oxygen saturation trend data and an illustrative severity index value in accordance with some embodiments of the present disclosure; and

FIG. 8 is an illustrative neural network for distinguishing between central apnea and obstructive apnea in accordance with some embodiments of the present disclosure.

### DETAILED DESCRIPTION OF THE FIGURES

A patient monitoring system may receive a physiological signal such as a photoplethysmograph (PPG) signal. Physiological parameters, including oxygen saturation, may be calculated from the physiological signal. The oxygen saturation data may be stored as time series of oxygen saturation data at a regular interval such as once every second. The time series of oxygen saturation data may include information related to the respiration of a monitored patient and may be used to determine the presence of a ventilatory instability. For example, certain patterns or clustering of patterns in the time series of oxygen saturation data may be indicative of the presence of a ventilatory instability. A ventilatory instability may include any one or more physiological conditions, events, or both. For example, one suitable type of ventilatory instability is apnea.

While ventilatory instability may be any one of a number of indications of patient airflow, for purposes of brevity and clarity the indication of patient airflow may be apnea. Apnea types include obstructive apnea and central apnea. Obstructive apnea may typically be caused by physical blockage of a patient's airway by human tissue, while central apnea may typically be the result of neurological issues that prevent proper respiratory function during sleep. As will be described herein, obstructive apnea and central apnea may impact respiration differently, such that obstructive apnea and central apnea may be identified and distinguished based on physiological information such as oxygen saturation data.

Once a pattern for ventilatory instability is detected, the patient monitoring system may also examine other recent occurrences of the pattern to determine whether the quantity and frequency of the occurrence of the pattern is sufficient to determine that a clustering state exists. If a clustering state exists, the patient monitoring system may calculate an index that is indicative of the severity of the apnea. The patient monitoring system may distinguish between types of apnea such as central apnea and obstructive apnea based on an analysis of predetermined metrics. The metrics may be calculated based on the set of patterns associated with the clustering state as well as on oxygen saturation time series data not included in the patterns. For example, the metrics may be input to a neural network, the output of which provides an indication of the likelihood that the detected apnea is a central apnea or that it is an obstructive apnea.

For purposes of clarity, the present disclosure is written in the context of the physiological signal being a PPG signal generated by a pulse oximetry system. It will be understood that any other suitable physiological signal or any other suitable system may be used in accordance with the teachings of the present disclosure.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I₀=intensity of light transmitted;
S=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I_{O}−(sβₒ+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for S yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for S using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

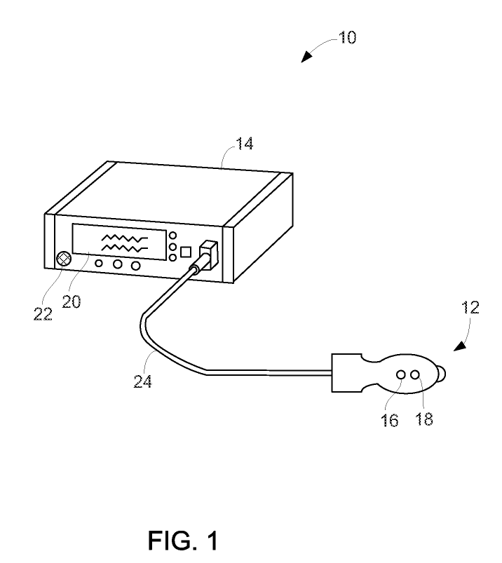

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation (e.g., SpO₂), and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

As is described herein, monitor 14 may analyze time series of oxygen saturation data to identify one or more physiological conditions. Although it will be understood that any suitable physiological conditions may be identified based on a time series of oxygen saturation data, in an exemplary embodiment, monitor 14 may identify physiological conditions related to respiration based on oxygen saturation data. For example, monitor 14 may detect ventilatory instability by analyzing a time series of oxygen saturation data. Detection of ventilatory instability is further described in U.S. patent application Ser. No. 12/388,114, U.S. patent application Ser. No. 12/409,688, U.S. patent application Ser. No. 12/609,304, U.S. patent application Ser. No. 12/609,314, and U.S. patent application Ser. No. 12/609,344, each of which is incorporated by reference herein in its entirety.

In some embodiments, monitor 14 may detect predefined patterns in the oxygen saturation time series data. These patterns or clusters of these patterns may be indicative of the presence of ventilatory instability. Moreover, these patterns or clusters of these patterns may be more specifically indicative of the presence of apnea. In some embodiments, monitor 14 may analyze certain characteristics of these patterns or clusters of these patterns to distinguish between the presence of central apnea and obstructive apnea.

In an exemplary embodiment, the analysis of a time series of oxygen saturation data may be performed based on samples of oxygen saturation data stored in memory. For example, oxygen saturation values may be determined as described above at any regular interval such as once every second. Although it will be understood that any suitable storage interval may be used, a storage interval such as once a second may be chosen to provide sufficient resolution without occupying excess memory based on the particular implementation.

The analysis of the time series of oxygen saturation data may be performed based on a window of samples of the time series of oxygen saturation data stored in memory. As is described herein, the time series of oxygen saturation data may be analyzed by monitor 14 to determine the presence of ventilatory instability, which for purposes of this disclosure is considered to be apnea, and to distinguish between of the presence of central apnea and obstructive apnea. However, it will be understood that the time series of oxygen saturation data could be transmitted to any suitable device for analysis, such as a local computer, a remote computer, a nurse station, mobile devices, tablet computers, or any other device capable of sending and receiving data and performing processing operations. Information may be transmitted from monitor 14 in any suitable manner, including wireless (e.g., WiFi, Bluetooth, etc.), wired (e.g., USB, Ethernet, etc.), or application-specific connections. The receiving device may analyze time series of oxygen saturation data as described herein.

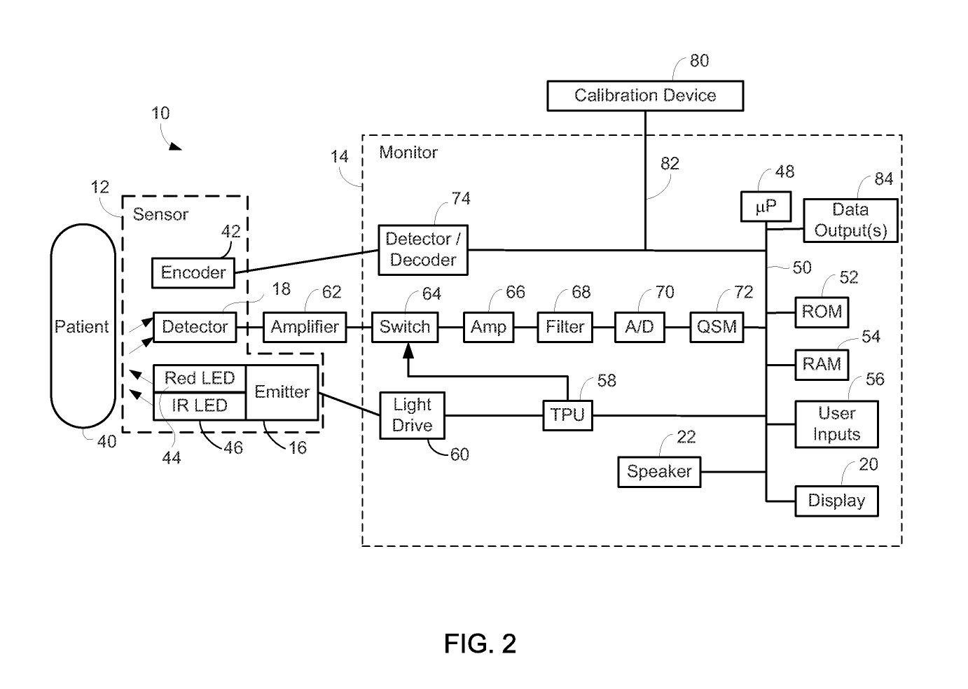

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42. In another example, a time series of oxygen saturation data may be classified based in part on information that may be stored in encoder 42 such as a sensor type, a patient's physiological characteristics (e.g., gender, age, weight), treatment information (e.g., that the patient is on supplemental oxygen), the type of sensor unit, or any combination thereof.

Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics and treatment information. In some embodiments, encoder 42 may include a memory on which one or more of the following information may be stored for communication to monitor 14; the type of the sensor unit 12; the wavelengths of light emitted by emitter 16; the particular wavelength each sensor in the sensor array is monitoring; a signal threshold for each sensor in the sensor array; any other suitable information; physiological characteristics (e.g., gender, age, weight); treatment information (e.g., that the patient is on supplemental oxygen); or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as oxygen saturation, pulse rate, and/or respiration information, using various algorithms and/or look-up tables based on the value of the received signals and/or data corresponding to the light received by detector 18. As described herein, microprocessor 48 may generate a time series of oxygen saturation data from determined oxygen saturation values, and determine one or more classifications based on the time series of oxygen saturation data. Microprocessor 48 may also set one or more flags or indicators based on an analysis of the received signals from sensor 12, determined values such as SpO₂ oxygen saturation and pulse rate, and patterns of data (e.g., oxygen saturation and pulse rate data). Although it will be understood that any suitable flags or indicators may be set, exemplary flags or indicators include an artifact flag, an invalid sample flag, and sensor status flags. An artifact flag may be a flag associated with samples of the PPG signal where it is determined that an artifact (e.g., a motion artifact) occurred. An invalid sample flag may be a flag associated with samples of the PPG signal where it is determined that the samples were invalid (e.g., due to measurement or calculation error). A sensor status flag may be a flag associated with samples of the PPG signal where it is determined that the sensor was disconnected or incorrectly connected to a patient. In an exemplary embodiment, the flags may be associated with a time series of oxygen saturation data, and may be stored along with the time series of data in RAM 54.

Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable microprocessor 48 to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include time series of oxygen saturation data and other related data (e.g., one or more flags) to be transmitted to an external device for determining one or more classifications based on the time series of oxygen saturation data.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

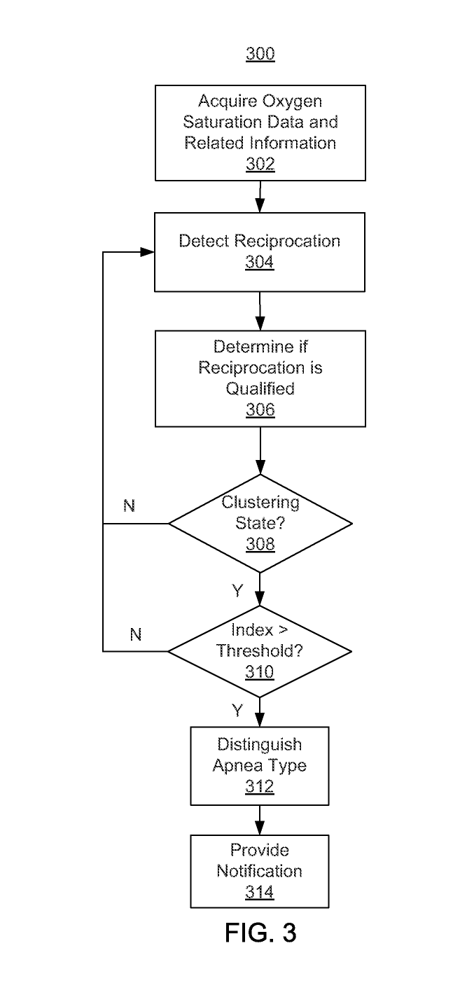

FIG. 3 is a flow diagram showing illustrative steps for analyzing a time series of oxygen saturation data in accordance with some embodiments of the present disclosure. In an exemplary embodiment, the steps described in FIG. 3 and related figures may be performed by system 10. However, it will be understood that some or all of the steps of FIG. 3 may be determined by one or more other devices such as a remote or networked monitor.

Oxygen saturation measurements for a patient may change based on respiration or other physiological processes. In accordance with an exemplary embodiment, patterns of oxygen saturation data may be observed and analyzed over time to identify the occurrence of respiratory or other physiological processes. Although any suitable physiological processes may be identified based on patterns of time series of oxygen saturation data, in an exemplary embodiment, the system 10 may identify a pattern that is indicative of a ventilatory instability, which for purposes of this disclosure is reflective of the presence of apnea. In an exemplary embodiment, this pattern may be a potential apneic event (also referred to herein as a reciprocation). The pattern may be further analyzed to distinguish whether the apneic event is due to central apnea or obstructive apnea. An index may be generated that is indicative of the apneic event, central apnea, or obstructive apnea. Although the potential apneic events may be analyzed in any suitable manner, in an exemplary embodiment as described in FIG. 3, a series of metrics may be generated based on the time series of oxygen saturation data and other relevant information about the patient, and the metrics may be analyzed by one or more neural networks (e.g., a first neural network to identify ventilatory instability, and a second neural network to distinguish central apnea from obstructive apnea).

Referring to FIG. 3, at step 302 system 10 acquires a time series of oxygen saturation and related data. The time series of oxygen saturation data may be acquired, stored, and analyzed in any suitable manner. In an exemplary embodiment, microprocessor 48 may calculate oxygen saturation values on a periodic basis based on information received and processed by monitor 14 from sensor 12. Oxygen saturation values may be calculated and stored at any suitable intervals such as once every second. In some embodiments, oxygen saturation data may be provided as part of a previously stored file of physiological data or may be provided in real-time from a separate monitoring device communicatively coupled to monitor 14. Additional related information such as an artifact flag, invalid sample flag, and sensor status flags may also be determined and may be associated with oxygen saturation values. The oxygen saturation values and related information may be stored, e.g., in RAM 54. Although the time series of oxygen saturation data and related information may be accessed by microprocessor 48 for any suitable purpose, in an exemplary embodiment, the time series of oxygen saturation data may be accessed by microprocessor 48 to identify ventilatory instability and distinguish between central apnea and obstructive apnea.

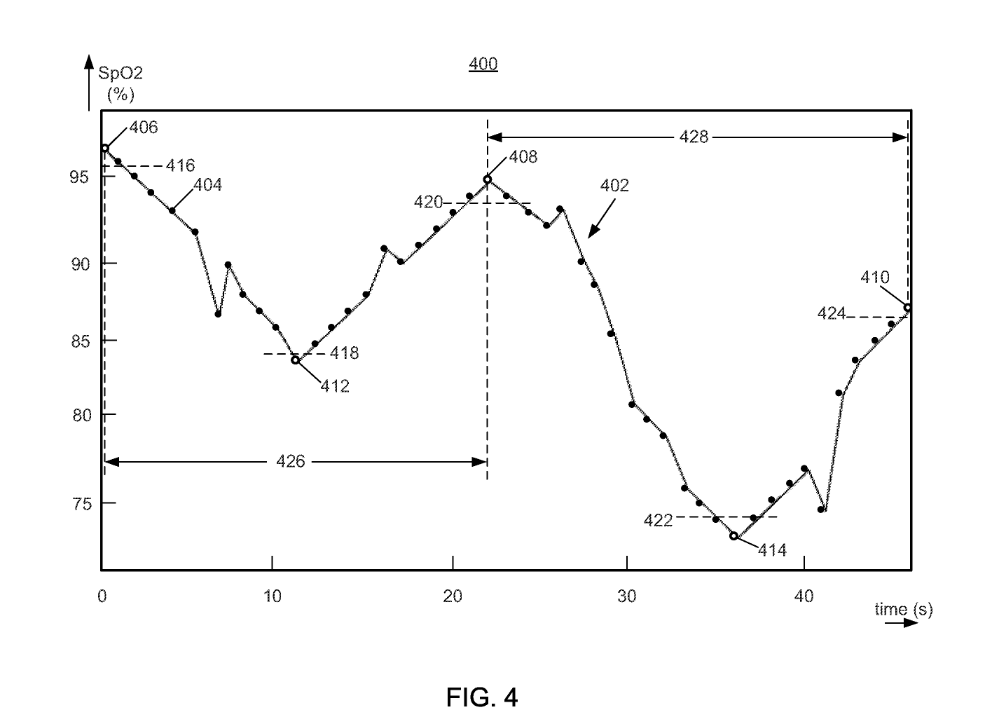

At step 304, microprocessor 48 may detect one or more events including a desaturation (drop in oxygen saturation value) that is followed by a resaturation (rise in oxygen saturation value) based on the time series of oxygen saturation data. For purposes of this disclosure a desaturation/resaturation event may be referred to as a potential apneic event or a reciprocation. A reciprocation may occur when the degree of change of the time series of oxygen saturation data exceeds one or more thresholds over a period of time. A graph 400 of exemplary reciprocations is depicted in FIG. 4. The ordinate of graph 400 may be in units of percentage oxygen saturation concentration, the abscissa may be in units of seconds, and the underlying time series of oxygen saturation data 402 may have been stored at one second intervals as is depicted by points 404. As will be described herein, a subset of points 404 may correspond to peaks (i.e., peak 406, peak 408, and peak 410) and another subset of points 404 may correspond to nadirs (i.e., nadir 412 and nadir 414).

Although a reciprocation may be detected in any suitable manner, in an exemplary embodiment, a reciprocation may be defined by a fall peak, a nadir, and a rise peak. A peak may be a maximum oxygen saturation value of a segment of the time series of oxygen saturation data that exceeds an upper threshold band. A fall peak may define the beginning of a potential reciprocation, while a rise peak may define the end point of a potential reciprocation. In some instances, a single peak may serve as both a rise peak (defining the end of a first reciprocation) and a fall peak (defining the beginning of a second reciprocation). A nadir may be a minimum oxygen saturation value of a segment of a time series of oxygen saturation data that is less than a lower threshold band. Although a reciprocation may be detected in any suitable manner, in an exemplary embodiment, a reciprocation may be any segment of oxygen saturation data in which a fall peak, nadir, and rise peak occur in sequence.

As is depicted in FIG. 4, a series of threshold values 416, 418, 420, 422, and 424 may be established as will be described in more detail herein. Peak 406 may be a maximum value that exceeds an upper threshold 416, nadir 412 may be a minimum value that is less than lower threshold 418, peak 408 may be a maximum value that exceeds upper threshold 420, nadir 414 may be a minimum value that is less than lower threshold 422, and peak 410 may be a maximum value that is greater than upper threshold 424. In an exemplary embodiment, peak 406, nadir 412, and peak 408 may define reciprocation 426, with peak 406 corresponding to a fall peak and peak 408 corresponding to a rise peak. Peak 408, nadir 414, and peak 410 may define reciprocation 428, with peak 408 corresponding to a fall peak and peak 410 corresponding to a rise peak.

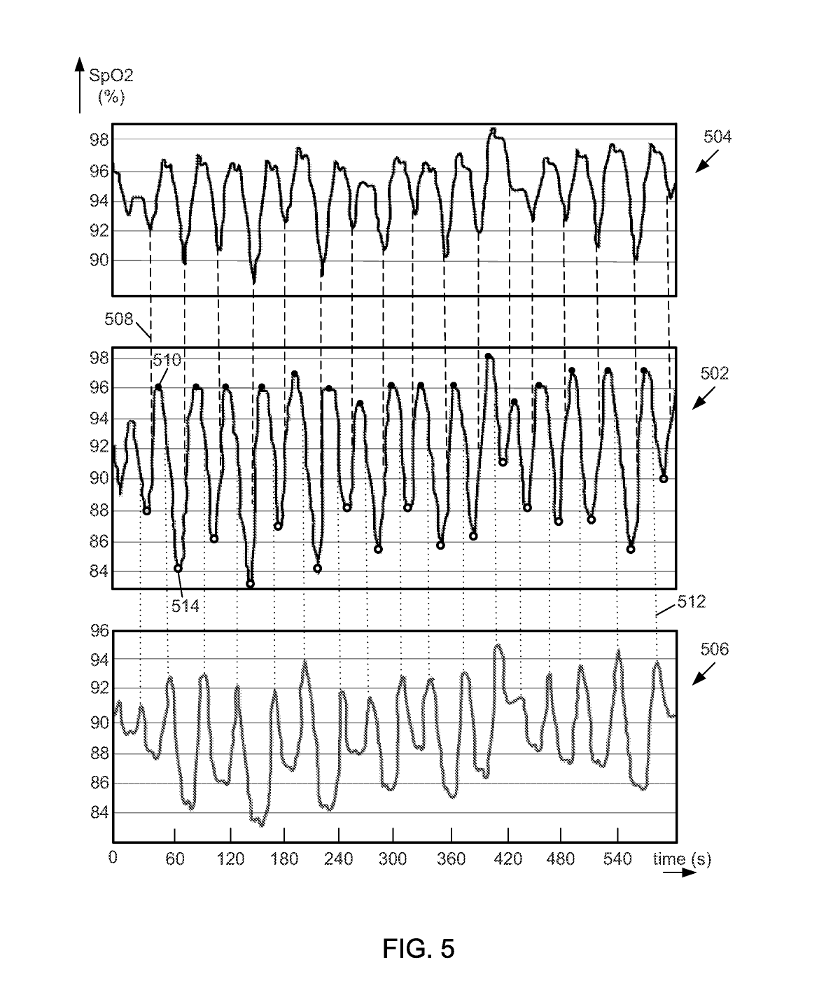

FIG. 5 is a series of graphs depicting an exemplary time series of oxygen saturation data 502, an exemplary upper threshold signal 504, and an exemplary lower threshold signal 506 in accordance with some embodiments of the present disclosure. Although thresholds for identifying reciprocations from a time series of oxygen saturation data 502 may be determined in any suitable manner, in an exemplary embodiment, the threshold values may be updated on a rolling basis based on the underlying time series of oxygen saturation data 502. In an exemplary embodiment, upper threshold signal 504 and lower threshold signal 506 may be generated based at least in part on a mean and standard deviation of the time series of oxygen saturation data 502. The values for upper threshold signal 504 may be calculated on a periodic basis (e.g., once per second, corresponding to each individual value of the time series of oxygen saturation data) as the rolling mean of a subset of the time series of oxygen saturation data 502 plus the rolling standard deviation of the subset of the time series of oxygen saturation data 502. The values for lower threshold signal 506 may be calculated on a periodic basis (e.g., once per second, corresponding to each individual value of the time series of oxygen saturation data) as the rolling mean of a subset of the time series of oxygen saturation data 502 minus the rolling standard deviation of the subset of the time series of oxygen saturation data 502.

Although the subset of the time series of oxygen saturation data 502 used to calculate values of the upper threshold signal 504 and the lower threshold signal 506 may be determined in any suitable manner, in an exemplary embodiment, an initial subset of the most recent 12 seconds of oxygen saturation data (e.g., corresponding to 12 values of the time series of oxygen saturation data 502) may be used. This initial subset of the time series of oxygen saturation data 502 may be used in conditions when no reciprocations have been detected or when a large amount of time (e.g., six minutes) has passed since the previous reciprocation. If a reciprocation has occurred recently (e.g., within the previous six minutes) the subset of the time series of oxygen saturation data 502 used to calculate the values for the upper threshold signal 504 and lower threshold signal 506 may be based on the duration of the current reciprocation and/or the duration of one or more preceding reciprocations as follows:

duration=½*current+½*previous (14)

where:
duration=subset of the time series of oxygen saturation data for determining thresholds;
current=duration of current reciprocation; and
previous=duration of previous reciprocation.

In this manner, the subset of data used to calculate values of upper threshold signal 504 and lower threshold signal 506 may vary based on the duration of the most recent reciprocations. In other exemplary embodiments, the duration may also be based on other factors such as a response mode for the pulse oximetry algorithm (e.g., how frequently data is processed), artifact or signal quality metrics, or the sensor type. In an exemplary embodiment, a maximum and/or minimum duration may be established. An exemplary value for a minimum duration may be 12 seconds and an exemplary value for a maximum duration may be 36 seconds.

In an exemplary embodiment, upper threshold signal 504 and lower threshold signal 506 may be determined from values of the time series of oxygen saturation data 502 as described above. The ordinate of the graphs of FIG. 5 may be in units of percentage oxygen saturation and the abscissa of the graphs of FIG. 5 may be in units of seconds. Dashed lines 508 may indicate points at which the value of the time series of oxygen saturation data 502 crosses the value of upper threshold signal 504 on an upward slope. Peaks 510 may correspond to maximum values of the time series of oxygen saturation data 502 following each respective crossing of upper threshold signal 504.

Dotted lines 512 may indicate points at which the value of the time series of oxygen saturation data 502 crosses the value of lower threshold signal 506 on a downward slope. Nadirs 514 may correspond to minimum values of the time series of oxygen saturation data 502 following each respective crossing of lower threshold signal 506. Each peak 510 may be a fall peak that defines the beginning of a potential reciprocation, and a reciprocation may be detected if a nadir 514 occurs prior to the next rise peak (i.e., the next peak 510).

Referring again to FIG. 3, once one or more potential apneic events or reciprocations are detected at step 304 processing may continue to step 306 to qualify the one or more reciprocations. Although it will be understood that reciprocations can be qualified in any suitable manner, in an exemplary embodiment, a series of metrics may be calculated for each reciprocation, the metrics may be input to a neural network, and an output value from the neural network may indicate whether a particular reciprocation (potential apneic event) is a qualified apneic event.

Although it will be understood that any suitable metrics may be calculated, in an exemplary embodiment, a unique subset of metrics may be determined to analyze the reciprocation. For example, a set of metrics may be determined for the reciprocation to qualify or otherwise analyze the reciprocation. Although any suitable metrics may be used to perform any suitable analysis, in an exemplary embodiment, a unique set of metrics is used to qualify the reciprocation as exhibiting ventilatory instability (e.g., determining whether a detected reciprocation is the result of physiological processes) and another unique set of metrics is used to determine a type of apnea (e.g., central apnea or obstructive apnea). In an exemplary embodiment, the following metrics may be used to qualify the reciprocation as being due to ventilatory instability: fall slope, rise slope, motion percentage, magnitude, slope ratio, path length ratio, peak difference, a number of consecutive reciprocations metric, a maximum value metric, artifact percentage, slope ratio difference, duration difference, nadir difference, and path length ratio difference. In an exemplary embodiment the metrics used to distinguish central apnea from obstructive apnea may include any or all of the metrics described above as well as any or all of the following metrics: a magnitude ratio metric, a change in magnitude ratio metric, a relative change in peak metric, a relative change in nadir metric, a pulse rate metric, a percent modulation metric, a frequency content metric, a standard deviation of the oxygen saturation metric, and a patient information metric. Each of these metrics is described below.

An exemplary metric may include a motion percentage over the current reciprocation. The motion percentage is based on the number of the samples of the reciprocation that are flagged with an artifact flag divided by the total number of samples in the reciprocation. Another related metric may be the motion percentage for one or more of the previous reciprocations.

Another exemplary metric may be a path length ratio for the current reciprocation. The path length is the summation of the current oxygen saturation value minus the previous oxygen saturation value for all oxygen saturation values in a reciprocation. The path length ratio may be determined from the path length as follows:

PLratio=PL/((Fpeak−Nadir)+(Rpeak−Nadir)) (15)

where:
PLratio=path length ratio for a reciprocation;
PL=path length associated with the reciprocation;
Fpeak=oxygen saturation value of fall peak for the reciprocation;
Nadir=oxygen saturation value of nadir for the reciprocation; and
Rpeak=oxygen saturation value of rise peak for the reciprocation.

Another exemplary metric may be a rise slope for a reciprocation. The rise slope is the oxygen saturation value of the rise peak minus the oxygen saturation value of the nadir, with the result divided by the time between the nadir and the rise peak.

Another exemplary metric may be a fall slope for a reciprocation. The fall slope is the oxygen saturation value associated with the nadir minus the oxygen saturation value associated with the fall peak, with the result divided by the time between the fall peak and the nadir.

Another exemplary metric may be the slope ratio for a reciprocation. The slope ratio is any ratio between fall slope and the rise slope. In an exemplary embodiment, the slope ratio may be the fall slope divided by the rise slope.

Another exemplary metric may be a change in slope ratio between the previous reciprocation and the current reciprocation. In another embodiment, the change in slope ratio may be based on the difference between the slope ratio for the current reciprocation and the slope ratio of the last qualified reciprocation.

Another exemplary metric may be a magnitude of a reciprocation. The magnitude is the greater of the oxygen saturation values of the fall peak and rise peak, minus the oxygen saturation value of the nadir.

Another exemplary metric may be a magnitude ratio of a reciprocation. The magnitude ratio is the ratio of the magnitude of the fall peak (i.e., the oxygen saturation value of the fall peak minus the oxygen saturation value of the nadir) over the magnitude of the rise peak (i.e., the oxygen saturation value of the rise peak minus the oxygen saturation value of the nadir) for a reciprocation. In another embodiment, the magnitude ratio may be the magnitude of the rise peak over the magnitude of the fall peak.

Another exemplary metric may be a change in the magnitude ratio for a reciprocation. The change in magnitude ratio is based on the difference between the magnitude ratio of the current reciprocation and the magnitude ratio of the previous reciprocation. In another embodiment, the change in the magnitude ratio may be based on the magnitude ratio of the current reciprocation and the magnitude ratio of the last qualified reciprocation.

Another exemplary metric may be a peak difference of a reciprocation. The peak difference is the absolute value of the difference between the fall peak and the rise peak for a reciprocation.

Another exemplary metric may be a number of consecutive reciprocations metric. The number of consecutive reciprocations metric is a count of the number of consecutive reciprocations that have: (1) met predetermined limits imposed for the fall slope, magnitude, slope ratio, and path length ratio metrics defined above; and (2) are less than or equal to 120 seconds apart. The number of consecutive reciprocations value is reset to 0 whenever the gap between any two detected reciprocations exceeds 120 seconds. The number of consecutive reciprocations metric is limited to a maximum value determined by a constant that represents a maximum number of qualified reciprocations for the measurement period (e.g., 120 seconds). The value for the maximum number of qualified reciprocations be modified based on a response mode of system 10. In an exemplary embodiment, this value is 10 in normal response mode and 15 in fast response mode.

Another exemplary metric may be a maximum value metric. The maximum value metric measures the maximum value for a reciprocation and is the greater of the oxygen saturation value of the fall peak and the oxygen saturation value of the rise peak.

Another exemplary metric may be an artifact percentage for a reciprocation. The artifact percentage is the percentage of the oxygen saturation values within a reciprocation that are associated with an artifact flag.

Another exemplary metric may be a duration difference for a reciprocation. The duration difference is based on the difference between the duration of the current reciprocation and the previous reciprocation. In another embodiment, the duration difference may be based on the difference between the duration of the current reciprocation and the duration of the last qualified reciprocation. Another exemplary metric may be a percentage change in duration between the current reciprocation and the previous reciprocation or last qualified reciprocation.

Another exemplary metric may be a nadir difference for a reciprocation. The nadir difference is based on the difference between the oxygen saturation value of the nadir of the current reciprocation and the nadir of the previous reciprocation. In another embodiment, the nadir difference may be based on the oxygen saturation value of the nadir of the current reciprocation and the nadir of the last qualified reciprocation. Another exemplary metric may be a percentage change in the oxygen saturation value between the nadir of the current reciprocation and the nadir of the previous reciprocation or last qualified reciprocation.

Another exemplary metric may be a path length ratio difference for a reciprocation. The path length ratio difference is based on the difference between the path length ratio of the current reciprocation and the path length ratio of the previous reciprocation. In another embodiment, the path length difference may be based on the path length ratio of the current reciprocation and the path length ratio of the last qualified reciprocation.

Another exemplary metric may be a pulse rate associated with a reciprocation. The pulse rate may be associated with the current reciprocation and/or one or more previous reciprocations.

Another exemplary metric may be a percent modulation of the IR or Red wavelength signals from sensor 12. In an exemplary embodiment, the percent modulation may be considered to qualify or disqualify potential reciprocations. If the amplitude modulation is low for a potential reciprocation, such that an oximetry algorithm may have difficulty deriving an accurate oxygen saturation value, the reciprocation may be disqualified, or any reciprocation metrics down weighted, as part of the analysis. A similar approach may be taken for a high percent modulation. In an embodiment, the modulation of individual cardiac pulses may also be considered. Cardiac pulse amplitude modulation that increases or remains unchanged during a potential reciprocation may indicate that the subject is still trying to breathe, but there is no ventilation, which would signify a potential obstructive apnea. A significant decrease in cardiac pulse amplitude modulation during a reciprocation may indicate that there is no drive to breathe during the reciprocation, which may be indicative of a central apnea. Frequency and/or baseline modulation could also be used in a similar manner. In an embodiment, percent modulation may be a based on dividing the alternating (AC) portion of a plethysmograph signal (e.g., the IR Plethysmograph signal) by the constant (DC) portion of the plethysmograph signal. In some embodiments, the underlying signal may be filtered and scaled.

Another exemplary metric may be a frequency content of the pulse rate or the time series of oxygen saturation data. Although the frequency content may be determined in any suitable manner, in an exemplary embodiment, the frequency content may be based on finding a prominent peak in the autocorrelation of the pulse rate or oxygen saturation data time series. Any suitable method may be used for detecting a prominent frequency, such as a continuous wavelet transform or fast Fourier transform. In some embodiments, if only the gross estimate of frequency is utilized, the system may determine a gross (or “average”) frequency content of a time series by comparing the power of a time series to the power of its derivative:

Frequency Content(Hz)=(1/(2πdt))*std(x′)/std(x) (16)

x=input time series
x′=derivative of the time series
dt=time series sample interval
std=standard deviation operation (an estimate of signal power)

Another exemplary metric may be a standard deviation of the time series of oxygen saturation data calculated by a peak detection technique. In an exemplary embodiment, reciprocations or sections of SpO2 trend with very large standard deviations beyond the bounds of what is considered clinically possible may be disqualified, or metrics from the reciprocations with excessively high variability may be down-weighted in an analysis.

A number of the exemplary metrics described herein are based on the difference between the current reciprocation and a previous reciprocation. For each of these exemplary metrics any number of previous reciprocations could be factored into the difference calculation. In an exemplary embodiment, a mean, median, or standard deviation of the difference values may be determined as an exemplary metric.

Although a number of exemplary metrics have been described herein, it will be understood that any suitable metric value may be used as an input to qualify and/or classify the time series of oxygen saturation data. A suitable metric may include any information relating to a patient, patient treatments, or the patient's physiological condition. Other exemplary metrics include patient gender, age, weight, or indications of treatments such as whether the patient is on supplemental oxygen. Any flag (e.g., artifact flag, invalid sample flag, and sensor status flags) or physiological values (e.g., oxygen saturation values, pulse rate values, blood pressure values, respiration values) may also be used to assist in determining metrics (e.g., based on a percentage of a reciprocation associated with the flag or an out-of-range physiological value).

Once the relevant metrics have been calculated, the metrics may be used to determine whether the reciprocation (potential apneic event) is a qualified reciprocation (qualified apneic event). A qualified apneic event may be determined in any suitable manner from the metrics of interest, including using a linear qualification function or a trained neural network. In an exemplary embodiment, a neural network may be generated to qualify the qualified apneic event for ventilatory instability. A neural network may be generated based on a set of training data associated with ventilatory instability. The set of metrics relevant to qualify the reciprocation, as well as the coefficients and transfer function associated with the neural network, may be determined based on the training data.

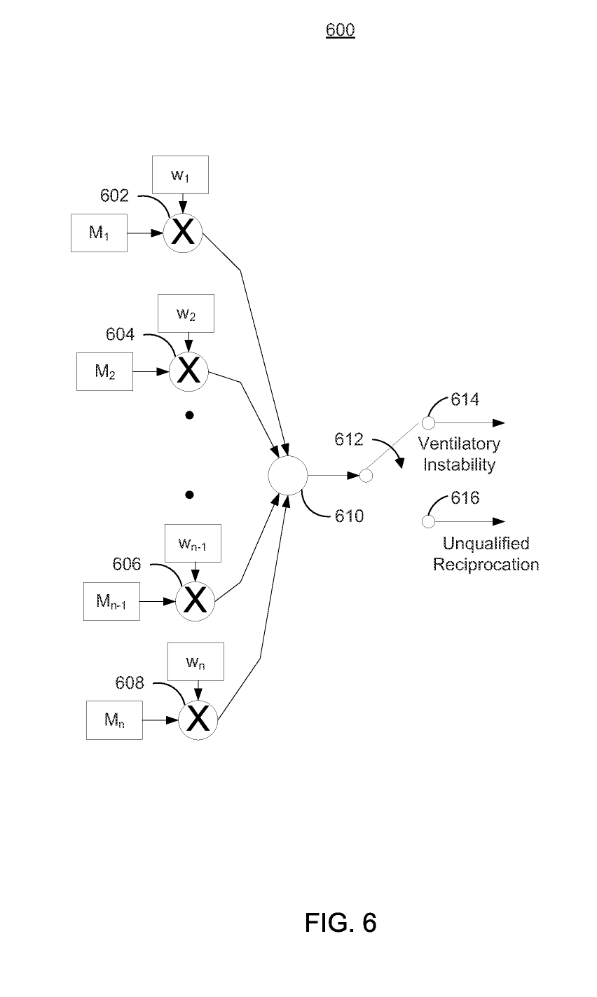

FIG. 6 depicts a neural network 600 for determining whether a detected reciprocation from step 304 is a qualified reciprocation at step 306. The inputs to neural network 600 may include a series of n metrics M₁, M₂, . . . Mₙ₋₁, Mₙ. The n metrics may be metrics associated with qualifying a reciprocation as described herein. Although any suitable metrics may be used to qualify a reciprocation for ventilatory instability, in an exemplary embodiment the metrics may include the following: fall slope, magnitude, slope ratio, path length ratio, peak difference, the number of consecutive reciprocations metric, a maximum value metric, artifact percentage, slope ratio difference, duration difference, nadir difference, and path length ratio difference. A series of n coefficients w₁, w₂, . . . wₙ₋₁, wₙ may be associated with the metrics based on the results of the training data. At each of nodes 602, 604, . . . 606, and 608, each metric may be multiplied with its corresponding coefficient. The resulting values may be inputs to neuron 610 of neural network 600. Although neuron 610 may generate an output value from the inputs in any suitable manner, in an exemplary embodiment, neuron 610 may implement a linear transfer function to generate the output values. It will be understood that any suitable transfer function such as a log-sigmoid transfer function may be implemented by neuron 610. Any other suitable transfer function may be implemented such as, for example, a linear transfer function or a polynomial transfer function of any suitable order.

The output of neuron 610 may be compared to a threshold 612. Although the threshold may be any suitable value, in an exemplary embodiment, the threshold may be selected based on empirical data, desired sensitivity, or both. In an exemplary embodiment, if the value of the output of neuron 610 exceeds threshold 612, the reciprocation of interest may be a qualified apneic event indicative of ventilatory instability 614. If the output of neuron 610 does not exceed threshold 612, the reciprocation of interest may be considered to be unqualified 616.

Referring again to FIG. 3, at step 308 a set of recent reciprocations may be analyzed to determine whether a clustering state exists. A clustering state may indicate that a particular minimum number of qualified apneic events have been identified within a particular period of time. In an exemplary embodiment, an event counter may keep a cumulative count of the qualified apneic events based on a set of rules. If the value of the event counter exceeds a threshold (e.g., five), a clustering flag may be used to indicate a clustering state.

Although any suitable factors or parameters may be used to implement the event counter, in an exemplary embodiment, the rules may be based upon the occurrence of qualified apneic events and the elapsed time between qualified apneic events. In an exemplary embodiment, the event counter may initially be set to zero, and the event counter may not have a negative value (i.e., it may not be reduced below zero). If the current reciprocation is a qualified apneic event and the event counter is at zero, the event counter may be incremented. Once the event counter is set to a non-zero value, each subsequent reciprocation may result in a modification of the event counter value. The event counter may be incremented for each subsequent reciprocation that is a qualified apneic event and occurs within a temporal threshold (e.g., 120 seconds) of the previous qualified apneic event. The event counter may be reduced (e.g., by two) to a minimum of zero for each detected reciprocation that is not qualified. The event counter may be reset to zero if the elapsed time between qualified apneic events is greater than the temporal threshold (e.g., 120 seconds).

The clustering state flag may be set to active based on the value of the event counter. Although the criteria for the clustering state flag may be established in any suitable manner, in an exemplary embodiment, the flag may be set to active if the event counter exceeds a threshold (e.g., five) and the two most recent reciprocations are qualified apneic events. The clustering state flag may remain active while the value of the event counter equals or exceeds the threshold. If a clustering state exists, processing may continue to step 310. If a clustering state does not exist, processing may return to step 304.

At step 310, a severity index value may be calculated for the time series of oxygen saturation data when a clustering state exists. Although a severity index value for the time series of oxygen saturation data may be calculated in any suitable manner, in an exemplary embodiment, the severity index value may be calculated based on the clustering state and information relating to one or more qualified apneic events. Although a particular procedure for calculating a severity index value is described herein, it will be understood that any suitable procedure may be used, and further that the aspects of the procedure (e.g., coefficients, measured values, filtering parameters, etc.) may be modified in any suitable manner.

In an exemplary embodiment, an unfiltered value may be calculated for each qualified apneic event whenever the clustering state is active. The unfiltered value may be based on one or more measurements or metrics for the qualified apneic event that are relevant to the qualification or classification of interest. In this manner, the unfiltered value may be related to the severity of the condition indicated by the qualification or classification of interest. In an exemplary embodiment, the unfiltered value may be based on the magnitude, peaks and nadirs for one or more qualified apneic events:

UFvalue=a*Mag+b*PeakDelta+c*NadirDelta (17)

where:
UFValue=unfiltered value;
a, b, and c=constants;
Mag=average magnitude of all reciprocations over a fixed time period (e.g., six minutes);
PeakDelta=the difference between the average of the three highest qualified apneic event rise peaks of the last six minutes and the average of the three lowest qualified apneic event rise peaks of the last six minutes; and
NadirDelta=the difference between the average of the three highest qualified apneic event nadirs of the last six minutes and the average of the three lowest qualified apneic event nadirs of the last six minutes.

The unfiltered value may be stored. To the extent that future reciprocations are not qualified, and thus no new unfiltered value is calculated for those reciprocations, the stored unfiltered value may be accessed and used to generate index values for the unqualified apneic events.

The unfiltered value (whether calculated or accessed from storage) may then be filtered to generate the severity index value. Although the unfiltered value may be filtered in any suitable manner, in an exemplary embodiment, the filter may be an infinite impulse response (IIR) filter with a selected response time (e.g., 40 seconds):

Index=UFvalue/d+PIndex*(d−1)/d (18)

where:
Index=severity index value;
UFValue=unfiltered value;
d=constant (e.g., 40 seconds); and
PIndex=previously calculated severity index value.

If the severity index value exceeds a maximum value (e.g., 31), the severity index value may be set to the maximum value. In step 310, the severity index value may be compared to a threshold value to determine whether to provide a notification or alarm to a user. Although the threshold value may be set in any suitable manner, in an exemplary embodiment, system 10 may include multiple sensitivity settings, with each sensitivity setting having an associated threshold value. For example, a high sensitivity setting may have an index threshold value of six, a medium sensitivity setting may have a severity index value of 15, and a low sensitivity setting may have a severity index value of 24.

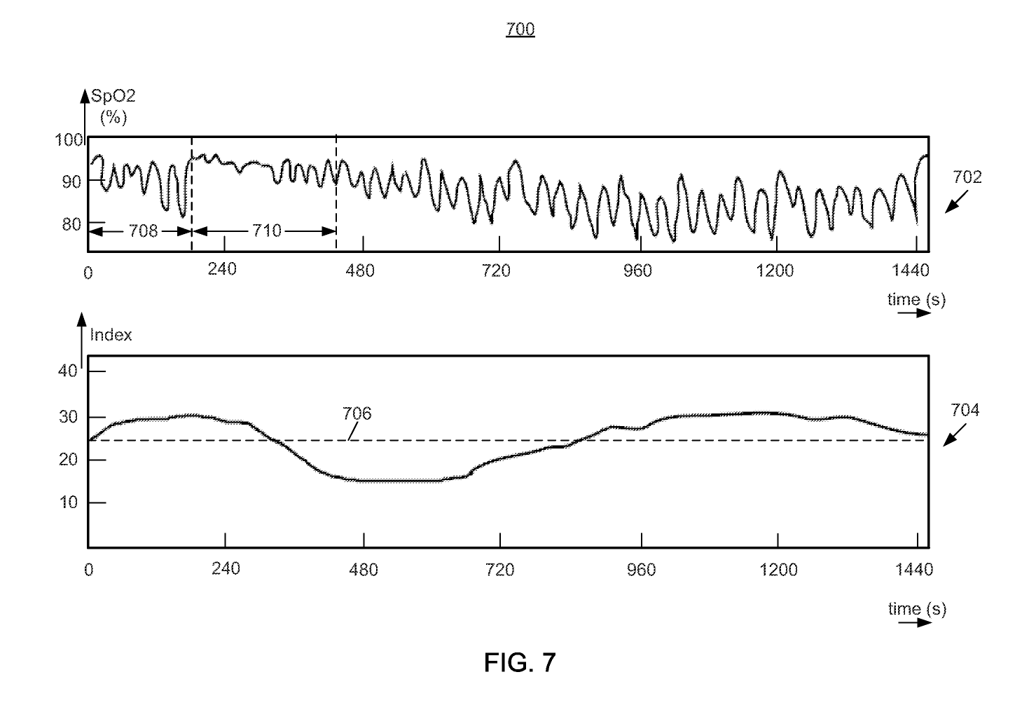

FIG. 7 depicts a set of graphs 700 of an exemplary time series of oxygen saturation data 702 and exemplary severity index values 704. The abscissa of each of graphs 702 and 704 may be in units of seconds, the ordinate of graph 702 may be in units of percentage oxygen saturation concentration (in this exemplary embodiment, SpO₂), and the ordinate of graph 704 may be the severity index value described herein. In the exemplary embodiment depicted in FIG. 7, the notification threshold 706 may correspond to a low sensitivity setting (e.g., a severity index value 24) for ventilatory instability.

At zero seconds, the severity index value may be approximately 24 which may result in a notification of ventilatory instability being provided by system 10. This notification may persist as long as the index exceeds the threshold. In a first region 708 (from approximately zero seconds to 190 seconds), the time series of oxygen saturation data 702 may include a number of qualified apneic events having a relatively high severity. The severity index value may therefore increase until it reaches the maximum value (e.g., 31). In a second region 710 (from approximately 190 to 440 seconds) the time series of oxygen saturation data 702 may include very few reciprocations and a majority of unqualified apneic events. The severity index value may therefore decrease and eventually drop below the notification threshold at approximately 320 seconds. The remaining portions the time series of oxygen saturation 702 data may again include a number of qualified apneic events having a relatively high severity. The severity index value may therefore again increase until it reaches the maximum value (e.g., 31), occasionally dropping below the maximum value based upon the severity of individual qualified apneic events.

In regions where the index exceeds the threshold, processing may continue to steps 312 and 314 to classify the ventilatory instability based on apnea type (e.g., central apnea vs. obstructive apnea) and provide a notification. In regions where the index does not exceed the threshold, processing may return to step 304.

At step 312, system 10 may determine a classification. Although any suitable classification may be determined, in an exemplary embodiment, the classification may distinguish between obstructive sleep apnea and central sleep apnea. The classification may be determined based on one or more metrics. Any suitable metrics may be used to determine the classification, such as those described herein. The metrics may be calculated for the most recent qualified reciprocation (i.e., the last reciprocation to result in the active clustering state), for all of the reciprocations associated with the currently active clustering state, for all of the qualified reciprocations associated with the currently active clustering state, for all of the reciprocations to occur within a predetermined or variable period of time, for all of the qualified reciprocations to occur within a predetermined or variable period time, any combination thereof, or based on any other suitable subset of the reciprocations or qualified reciprocations.

Although any suitable metrics may be used to distinguish central apnea from obstructive apnea, in an exemplary embodiment, the input metrics may include may include any or all of the metrics described with respect to the neural network of FIG. 6 as well as any or all of the following metrics: a magnitude ratio metric, a change in magnitude ratio metric, a relative change in peak metric, a relative change in nadir metric, a pulse rate metric, a percent modulation metric, a frequency modulation metric, a baseline modulation metric, a frequency content metric, a standard deviation of the oxygen saturation metric, and a patient information metric. In an exemplary embodiment, any metrics that are based on a single reciprocation may be calculated based on the most recent qualified reciprocation, while any metrics based on a plurality of reciprocations may be based on all of the qualified reciprocations to occur within the currently active clustering state.

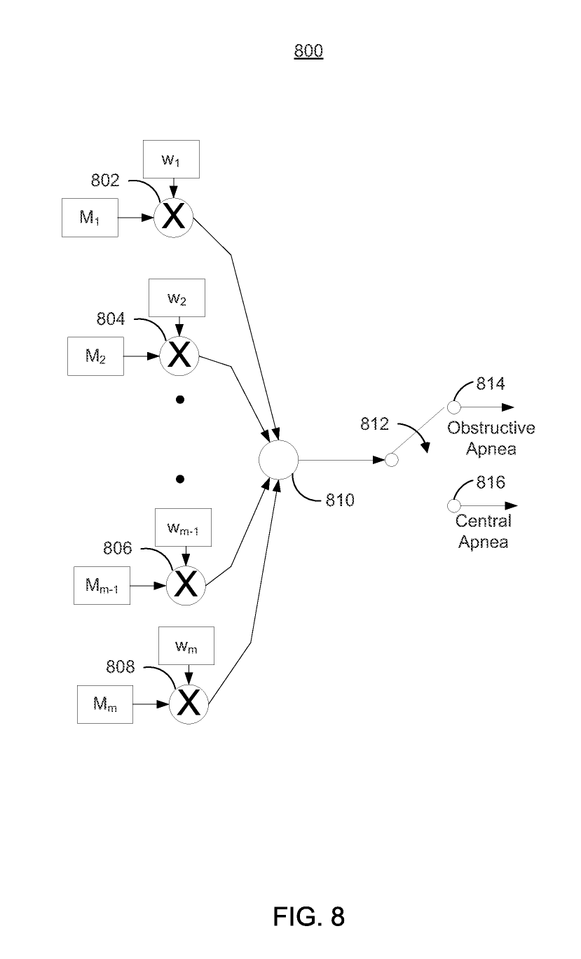

The calculated metrics may be inputs to a neural network which may calculate an output value that may be used to distinguish between central apnea and obstructive apnea. FIG. 8 depicts a neural network 800 for distinguishing between central apnea and obstructive apnea. The inputs to neural network 800 may include a series of m metrics M₁, M₂, . . . Mₘ₋₁, Mₘ as described herein.

A series of n coefficients w₁, w₂, . . . wₘ₋₁, wₘ may be associated with the metrics based on the results of the training data. At each of nodes 802, 804, . . . 806, and 808, each metric may be multiplied with its corresponding coefficient. The resulting values may be inputs to neuron 810 of neural network 800. Although neuron 810 may generate an output value from the inputs in any suitable manner, in an exemplary embodiment, neuron 810 may implement a linear transfer function to generate the output values. It will be understood that any suitable transfer function such as a log-sigmoid transfer function may be implemented by neuron 810. Any other suitable transfer function may be implemented such as, for example, a linear transfer function or a polynomial transfer function of any suitable order.

The output of neuron 810 may be compared to a threshold 812. Although the threshold may be any suitable value, in an exemplary embodiment, the threshold may be selected based on empirical data, desired sensitivity, or both. In an exemplary embodiment, if the value of the output of neuron 810 exceeds threshold 812, the ventilatory instability may be determined to be due to obstructive apnea 814. If the output of neuron 810 does not exceed threshold 812, the ventilatory instability may be determined to be due to central apnea 816.

Referring again to FIG. 3, at step 314 a notification may be provided based on the calculated severity index value and the classification of the apnea type. System 10 may provide any suitable notification to the user, such as an audible or visual alarm. System 10 may also display a message or transmit a message to a remote location such as a remote monitor or nurse station. Any suitable alarm or message may be provided to the user, such as a message that a limit for a particular classification has been exceeded (e.g., “obstructive apnea detected” or “central apnea detected”).

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method for classifying an apneic event, the method comprising:
detecting, using processing equipment, a potential apneic event in a time series of oxygen saturation data, the potential apneic event being in the form of a desaturation followed by a resaturation defined by a fall peak, nadir, and rise peak crossing respective thresholds;
qualifying, using the processing equipment, the potential apneic event as a qualified apneic event using a first plurality of metrics derived from a portion of the time series of oxygen saturation data that corresponds to the potential apneic event; and
classifying, using the processing equipment, the qualified apneic event as being due to one of central apnea and obstructive apnea based on the output of a neural network the inputs to which comprise at least the first plurality of metrics and a second plurality of metrics.

2. The method of claim 1, wherein the first plurality of metrics and the second plurality of metrics are selected from the group consisting of a fall slope metric, a magnitude metric, a slope ratio metric, a path length ratio metric, a peak difference metric, a number of consecutive reciprocations metric, a maximum value metric, an artifact percentage metric, a slope ratio difference metric, a duration difference metric, a nadir difference metric, a path length ratio difference metric, magnitude ratio metric, a change in magnitude ratio metric, a relative change in peak metric, a relative change in nadir metric, a pulse rate metric, a percent modulation metric, a frequency modulation metric, a baseline modulation metric, a frequency content metric, a standard deviation of the oxygen saturation metric, and a patient information metric, and any combination thereof.

3. The method of claim 1, wherein qualifying the potential apneic event comprises:
inputting the first plurality of metrics into a qualification neural network;
comparing an output of the qualification neural network to a threshold; and
determining whether to qualify the potential apneic event based on the comparing.

4. The method of claim 1, further comprising:
identifying a cluster of qualified apneic events occurring within a particular time period;
calculating a severity index value when the cluster is identified; and
providing an indication of the presence of a ventilatory instability based at least in part on the severity index value.

5. The method of claim 4 wherein providing the indication comprises triggering an alarm.

6. The method of claim 4 wherein providing the indication comprises providing an indication of the occurrence of an apneic episode, the indication indicating whether the episode is due to one of central apnea and obstructive apnea based at least in part on the classifying.

7. The method of claim 4, wherein identifying the cluster comprises:
determining a value for an event counter based on the qualified apneic event and one or more previous qualified apneic events occurring within the particular time period;
comparing the event counter to a threshold; and
identifying the cluster based on the comparing.

8. A non-transitory computer-readable storage medium for use in classifying an apneic event, the computer-readable medium having computer program instructions recorded thereon for:
detecting a potential apneic event in a time series of oxygen saturation data, the potential apneic event being in the form of a desaturation followed by a resaturation defined by a fall peak, nadir, and rise peak crossing respective thresholds;
qualifying the potential apneic event as a qualified apneic event using a first plurality of metrics derived from a portion of the time series of oxygen saturation data that corresponds to the potential apneic event; and
classifying the qualified apneic event as being due to one of central apnea and obstructive apnea based on the output of a neural network the inputs to which comprise at least the first plurality of metrics and a second plurality of metrics.

9. The computer-readable medium of claim 8, wherein the first plurality of metrics and the second plurality of metrics are selected from the group consisting of a fall slope metric, a magnitude metric, a slope ratio metric, a path length ratio metric, a peak difference metric, a number of consecutive reciprocations metric, a maximum value metric, an artifact percentage metric, a slope ratio difference metric, a duration difference metric, a nadir difference metric, a path length ratio difference metric, magnitude ratio metric, a change in magnitude ratio metric, a relative change in peak metric, a relative change in nadir metric, a pulse rate metric, a percent modulation metric, a frequency modulation metric, a baseline modulation metric, a frequency content metric, a standard deviation of the oxygen saturation metric, and a patient information metric, and any combination thereof.

10. The computer-readable medium of claim 8, wherein qualifying the potential apneic event comprises:
inputting the first plurality of metrics into a qualification neural network;
comparing an output of the qualification neural network to a threshold; and
determining whether to qualify the potential apneic event based on the comparing.

11. The computer-readable medium of claim 8, having further computer program instructions recorded thereon for:
identifying a cluster of qualified apneic events occurring within a particular time period;
calculating a severity index value when the cluster is identified; and
providing an indication of the presence of a ventilatory instability based at least in part on the severity index value.

12. The computer-readable medium of claim 11 wherein providing the indication comprises triggering an alarm.

13. The computer-readable medium of claim 11 wherein providing the indication comprises providing an indication of the occurrence of an apneic episode, the indication indicating whether the episode is due to one of central apnea and obstructive apnea based at least in part on the classifying.

14. A patient monitoring system comprising processing equipment configured to:
detect a potential apneic event in a time series of oxygen saturation data, the potential apneic event being in the form of a desaturation followed by a resaturation defined by a fall peak, nadir, and rise peak crossing respective thresholds;
qualify the potential apneic event as a qualified apneic event using a first plurality of metrics derived from a portion of the time series of oxygen saturation data that corresponds to the potential apneic event; and
classify the qualified apneic event as being due to one of central apnea and obstructive apnea based on the output of a neural network the inputs to which comprise at least the first plurality of metrics and a second plurality of metrics.

15. The patient monitoring system of claim 14, wherein the first plurality of metrics and the second plurality of metrics are selected from the group consisting of a fall slope metric, a magnitude metric, a slope ratio metric, a path length ratio metric, a peak difference metric, a number of consecutive reciprocations metric, a maximum value metric, an artifact percentage metric, a slope ratio difference metric, a duration difference metric, a nadir difference metric, a path length ratio difference metric, magnitude ratio metric, a change in magnitude ratio metric, a relative change in peak metric, a relative change in nadir metric, a pulse rate metric, a percent modulation metric, a frequency modulation metric, a baseline modulation metric, a frequency content metric, a standard deviation of the oxygen saturation metric, and a patient information metric, and any combination thereof.

16. The patient monitoring system of claim 14, wherein the processing equipment is further configured to:
input the first plurality of metrics into a qualification neural network;
compare an output of the qualification neural network to a threshold; and
determine whether to qualify the potential apneic event based on the comparison.

17. The patient monitoring system of claim 14, wherein the processing equipment is further configured to:
identify a cluster of qualified apneic events occurring within a particular time period;
calculate a severity index value when the cluster is identified; and
provide an indication of the presence of a ventilatory instability based at least in part on the severity index value.

18. The patient monitoring system of claim 17 wherein the indication comprises an alarm.

19. The patient monitoring system of claim 17 wherein the indication comprises an indication of the occurrence of an apneic episode, the indication indicating whether the episode is due to one of central apnea and obstructive apnea based at least in part on the classification.

20. The patient monitoring system of claim 17, wherein the processing equipment is further configured to:
determine a value for an event counter based on the qualified apneic event and one or more previous qualified apneic events occurring within the particular time period;
compare the event counter to a threshold; and
identify the cluster of qualified apneic events based on the comparison of the event counter to the threshold.
